(19) United States
(12) Patent Application Publication
(10) Pub. No.: US 2020/0267989 A1
(43) Pub. Date: Aug. 27, 2020

(54) PARASITE TREATMENT COMPOUND

(71) Applicant: University of Maine System Board of Trustees, Orono, ME (US)

(72) Inventors: Deborah A. Bouchard, Bradley, ME (US); Ian Bricknell, Hampden, ME (US)

(73) Assignee: University of Maine System Board of Trustees, Orono, ME (US)

(21) Appl. No.: 16/646,398

(22) PCT Filed: Sep. 12, 2018
(86) PCT No.: PCT/US2018/050663

§ 371 Date: Mar. 11, 2020

Related U.S. Application Data
(60) Provisional application No. 62/560,374, filed on Sep. 19, 2017.
(60) Provisional application No. 62/557,369, filed on Sep. 12, 2017.

(51) Int. Cl. A01N 59/08; A61K 33/14; A61P 33/14; A61K 35/08

(52) U.S. Cl. CPC A01N 59/08; A61K 35/08; A61P 33/14; A61K 33/14

(57) ABSTRACT

Methods for the removal of parasites from fish are disclosed, which comprise treatment with an aqueous solution comprising an ionic compound. Certain embodiments of the methods further comprise mechanical treatment.

### CROSS REFERENCE TO RELATED APPLICATION

This application claims the benefit of U.S. Application Ser. No. 62/557,369, filed Sep. 12, 2017 and to U.S. Application Ser. No. 62/560,374, filed on Sep. 19, 2017, the contents of both of which are hereby incorporated by reference in their entireties.

### BACKGROUND

Parasites reduce the host organism's health in many different ways. Parasites are generally much smaller than their host, and reproduce at a much faster rate. While attached to a host organism, a parasite reduces the host organism's health in many different ways including the modification of behavior and impairment of secondary sex characteristics. Meanwhile the parasites themselves improve their fitness by using the host organism for food, habitat and dispersal. The severity of harm done to a host organism depends greatly on the type of parasite and, in some instances, the number of parasites that attach to a single host. By way of specific example, sea lice are known to cause millions of dollars in damage to fish farms annually.

### SUMMARY OF THE DISCLOSURE

There is a need for a method for removing parasites attached to an infected fish using a composition, which is not toxic to fish, easy for fish farmers to handle, and environment friendly. The present invention, in various embodiments, provides methods for facilitating the removal of parasites attached to a fish.

In some embodiments, the present invention provides, inter alia, methods for facilitating removal a parasite attached to a fish including the step of treating the fish with an aqueous solution comprising an ionic compound, wherein the treating does not significantly harm or kill the fish.

In accordance with various embodiments, provided methods may be used to facilitate removal of a variety of aquatic parasites. In some embodiments, provided methods may be useful in removing any parasitic crustacean, parasitic aquatic arthropods, Platyhelminthes, and/or leeches. In some embodiments, the parasite is selected from the group consisting of sea lice, copepods, isopods, ostracods, aquatic mites, pycnogonids, cestodes, digeneans, and monogeneans. In some embodiments, provided methods may be useful in facilitating removal of protozoan parasites including those that cause amoebic gill disease (e.g., Neoparamoeba perurans).

In some embodiments, provided methods may be used to facilitate removal of at least one parasite from any variety of farmed fish (e.g., any fish suffering from one or more of the parasites listed above). In some embodiments, provided methods may be used to facilitate removal of parasites from any teleost fish useful in aquaculture as well as those being developed for future culture. For example, in some embodiments, the fish is a salmon, carp, tilapia, catfish, halibut, cod, swordfish, haddock, bass, grouper, trout, and/or tuna.

The present invention, in various aspects, includes the use of a variety of ionic compounds. For example, in some embodiments, the ionic compound is a potassium salt. In some embodiments, the ionic compound is a chloride salt. In some embodiments, the ionic compound is potassium chloride. In some embodiments, the aqueous solution comprises two or more ionic compounds.

In some embodiments, the present invention provides methods for facilitating removal of a parasite attached to a fish including the step of treating the fish with an aqueous solution comprising an ionic compound at a concentration of between about 1-5 ppt.

According to various embodiments, the treatment step may be of any application-appropriate length of time. In some embodiments, the step of treatment with an aqueous solution comprises placing the fish in the aqueous solution for about 60 minutes or less (e.g., 50, 40, 30, 20, 15, 10 minutes or less).

In some embodiments, the step of treatment with an aqueous solution comprises placing the fish in the aqueous solution for about 45 minutes or less. In some embodiments, the step of treatment with an aqueous solution comprises placing the fish in the aqueous solution for about 30 minutes or less. In some embodiments, the step of treatment with an aqueous solution comprises placing the fish in the aqueous solution for about 20 minutes or less. In some embodiments, the step of treatment with an aqueous solution comprises placing the fish in the aqueous solution for about 15 minutes or less. In some embodiments, the step of treatment with an aqueous solution is placing the fish in the aqueous solution for about 5 minutes.

Provided methods, according to various embodiments, include the use of a variety of aqueous solutions with provided ionic compounds. For example, in some embodiments, the aqueous solution comprises seawater. In some embodiments, the aqueous solution comprises freshwater.

It is specifically contemplated that the amount of ionic compound present in a particular aqueous solution will vary. By way of specific example, in some embodiments, an ionic compound is present in the aqueous solution at a concentration of about 100 ppt or less. In some embodiments, an ionic compound is present in the aqueous solution at a concentration of about 50 ppt or less. In some embodiments, an ionic compound is present in the aqueous solution at a concentration of about 25 ppt or less. In some embodiments, an ionic compound is present in the aqueous solution at a concentration of about 10 ppt or less. In some embodiments, an ionic compound is present in the aqueous solution at a concentration of about 5 ppt or less. In some embodiments, an ionic compound is present in the aqueous solution at a concentration of about 4 ppt. In some embodiments, an ionic compound is present in the aqueous solution at a concentration of about 3 ppt. In some embodiments, an ionic compound is present in the aqueous solution at a concentration of about 2 ppt. In some embodiments, an ionic compound is present in the aqueous solution at a concentration of about 1 ppt.

In some embodiments, it may be advantageous to include at least one additional mechanism for facilitating removal of a parasite from a fish. Accordingly, in some embodiments, provided methods further include a step of agitating a surface of the fish. In some embodiments, the step of agitating occurs prior to the step of treating the fish with the aqueous solution. In some embodiments, the step of agitating occurs substantially concurrently with the step of treating the fish with the aqueous solution. In some embodiments, the step of agitating occurs after the step of treating the fish with the aqueous solution. In some embodiments, the agitation of the fish occurs via mechanical means. In some embodiments, the mechanical means are or comprise scrubbing, shaking, and/or rolling. In some embodiments, the agitation of the fish occurs via a stream of a liquid. In some embodiments, the liquid is seawater, fresh water, the aqueous solution as used in the treatment step, or a mixture thereof. In some embodiments, the agitation occurs via a stream of a gas. In some embodiments, the gas is air.

In some embodiments, the present invention provides methods further including the steps of placing the fish in a controlled environment, and measuring the parasite concentration in the controlled environment both before treatment with the ionic compound and after treatment with the ionic compound.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF DRAWINGS

The foregoing and other objects, aspects, features, and advantages of the present disclosure will become more apparent and better understood by referring to the following description taken in conjunction with the accompanying figures in which:

FIG. 1 shows an illustration of a salmon (100) infected with a sea louse (110).

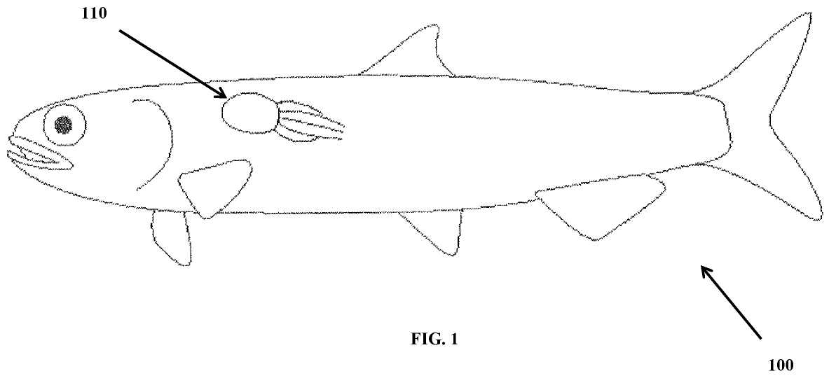

FIG. 2 shows a standard fish farm and a boat that contains a well-boat for treating the fish in the farm. As shown in the figure, a vacuum hose is used to extract the fish from the farm and place them in the well-boat. After the fish are treated in the well, the hose is reversed and the fish are returned to the farm.

### DEFINITIONS

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Chloride Salt: As used herein, the term “chloride salt” refers to any ionic compound comprising a chloride ion (Cr). A chloride salt can comprise a monoatomic cation or polyatomic cation. Examples of chloride salts include, but are not limited to, aluminum chloride, sodium chloride, potassium chloride, calcium chloride, ammonium chloride, magnesium chloride, and zinc chloride. in some embodiments, potassium chloride is a particularly advantageous potassium salt for use with certain provided methods. In some embodiments, a chloride salt does not include sodium chloride (NaCl).

Ionic Compound: As used herein, the term “ionic compound” refers to a chemical compound composed of ions held together by electrostatic forces termed ionic bonding. The ions can be monoatomic ions such as sodium (Na³⁰ ) and chloride (Cl⁻), or polyatomic ions such as ammonium (NH₄⁺) and carbonate (CO₃²⁻).

Parasite: As used herein, the term “parasite” refers to any parasite that appears on the external surface of a fish (skin, fins and gills) and is classed as an ecto-parasite. In some embodiments, the parasite can be monogenean parasites, such as skin flukes Gyrodactylus spp and gill flukes Dactylogyrus spp, protozoan parasites such as Ichthyophthirius multifiliis, Trichodina, Ichthyobodo (Costia), Chilodonella and Pleistophora, dinoflagellate Picinoodinium (Oodinium), aquatic crustacean parasites lernea (anchor worm) and Argulus, the larval stages of digenean parasites Clinostomum, Posthodiplostomum and Diplostomum, endoparasites worms (including cestodes and meatodes) including Spironucleus and Hexamita, or a mixture thereof. In some embodiments, the parasite is a sea louse (e.g., including those in the Lepophtheirus and Caligus genera, for example, Lepeophtheirus salmonis).

Potassium Salt: As used herein, the term “potassium salt” refers to any ionic compound comprising a potassium ion (K⁺). A potassium salt can comprise a monoatomic anion or polyatomic anion. Examples of potassium salts include, but are not limited to, potassium fluoride, potassium sulfide, potassium chloride, potassium nitride, potassium bromide, potassium iodide, potassium phosphate, potassium hydrogen phosphate, potassium dihydrogen phosphate, potassium sulfate, potassium nitrate, potassium hydrogen sulfate, potassium nitrite, potassium perchlorate, potassium iodate, potassium chlorate, potassium bromate, potassium chlorite, potassium hypochlorite, potassium hypobromite, potassium carbonate, potassium bicarbonate, potassium acetate, or potassium formate. In some embodiments, potassium chloride is a particularly advantageous potassium salt for use with certain provided methods.

Substantially: The term “substantially”, as used herein. refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION OF THE DISCLOSURE

The present invention is based, in part on the surprising realization that small amount of certain ionic compounds (e.g., potassium chloride, known to be lethal to fish in general and used to kill farm raised fish currently) are able to facilitate the removal of parasites from fish without causing substantial harm or death to the fish. In some embodiments, an indicator that a fish is suffering distress or substantial harm can be or comprise thrashing (e.g., motions similar to swimming upriver), loss of equilibrium and/or at least partial paralysis.

Provided Methods

Seawater comprises a number of salt ions. However, some salts are known to be toxic to fish. For example, U.S. Pat. No. 6,183,356 is directed to methods of using a potassium chloride injection as a means of causing a “humane” death in salmon.

Surprisingly, the present disclosure demonstrates that an aqueous solution comprising an ionic compound (e.g., potassium chloride) can be used to remove parasites attached to a fish. The Examples herein show that treatment of a fish with an aqueous solution of potassium chloride can remove sea lice in an effective and efficient manner, while having no material negative impact on the fish. Further, the present disclosure demonstrates that treatment of a fish with an aqueous solution comprising an ionic compound used in conjunction with other methods can significantly improve the effectiveness of parasite removal. Specifically, Example 5 demonstrates that a pre-treatment with an aqueous solution of potassium chloride followed by mechanical agitation removes parasites much more effectively than the mechanical treatment alone.

In some embodiments, the present invention provides, inter alfa, methods for facilitating removal a parasite attached to a fish including the step of treating the fish with an aqueous solution comprising an ionic compound, wherein the treating does not significantly harm or kill the fish. In some embodiments, treatment with an aqueous solution as described herein paralyzes parasites. In some embodiments, treatment with an aqueous solution as described herein kills parasites. In accordance with various embodiments, an ionic compound (e.g., potassium chloride) may be either agricultural or pharmaceutical grade.

In some embodiments, provided methods may be used to treat a population of fish where some of the fish are known to be infected/infested with parasites. In some embodiments, a method as described herein is used to treat a selected group of fish where each individual fish is known to be infected/infested with parasites. In some embodiments, provided methods may be used prophylactically to prevent or treat a suspected parasitic infection/infestation.

In some embodiments, a parasite is a sea louse. Sea lice are ecto-parasitic crustaceans that feed on the mucus, epidermal tissue, and blood of host marine fish. In some embodiments, a sea louse penetrates into a fish's tissue and causes illness and/or death. In some embodiments, a sea louse reduces the growth rate for a fish.

In some embodiments, an infected fish is a marine fish (e.g., lives in saltwater). In some embodiments, a fish is a freshwater fish. In some embodiments, a fish is a farm-raised fish. “Farm raised fish” include any fish that are living in a fish farm, where a fish farm is any suitable method of farming fish known in the art. In some embodiments, a fish is a farm-raised salmon.

In some embodiments, a step of treating an infected fish with an aqueous solution comprises placing the fish in the aqueous solution for a period of time (e.g., in a bath or a well-boat), which is sufficient to result in reducing the number of parasites attached to the fish without severely impacting the health of the fish. In some embodiments, an aqueous solution may be or comprise seawater. In some embodiments, a fish is treated with an aqueous solution which is not harmful to the health of the fish, and the fish is placed in the aqueous solution for a period of time, for example, about 5 hrs, about 4 hrs, about 3 hrs, about 2 hrs, or about 1-2 hrs. In some embodiments, a step of treating a fish with an aqueous solution comprises placing the fish in the aqueous solution for about 60 minutes or less. In some embodiments, a step of treating a fish with an aqueous solution comprises placing the fish in the aqueous solution for about 45 minutes or less. In some embodiments, a step of treating a fish with an aqueous solution comprises placing the fish in the aqueous solution for about 30 minutes or less. In some embodiments, a step of treating a fish with an aqueous solution comprises placing the fish in the aqueous solution for about 20 minutes or less. In some embodiments, a step of treating a fish with an aqueous solution comprises placing the fish in the aqueous solution for about 15 minutes or less. In some embodiments, a step of treating a fish with an aqueous solution comprises placing the fish in the aqueous solution for about 15 minutes. In some embodiments, a step of treating a fish with an aqueous solution comprises placing the fish in the aqueous solution for about 10 minutes or less. In some embodiments, a step of treating a fish with an aqueous solution comprises placing the fish in the aqueous solution for about 9 minutes, about 8 minutes, about 7 minutes, about 6 minutes, about 5 minutes, about 4 minutes, about 3 minutes, about 2 minutes, or about 1 minute

In some embodiments, an ionic compound is a chloride salt or a sulfate salt. In some embodiments, an ionic compound is a sodium salt, a magnesium salt (e.g. magnesium sulfate), a calcium salt, or a potassium salt.

In some embodiments, an ionic compound is a potassium salt. In some embodiments, a potassium salt is potassium chloride, potassium phosphate, potassium hydrogen phosphate, potassium dihydrogen phosphate, potassium carbonate, potassium bicarbonate, potassium sulfate, or potassium acetate.

In some embodiments, an ionic compound is a chloride salt. In some embodiments, a chloride salt is aluminum chloride, sodium chloride, potassium chloride, calcium chloride, ammonium chloride, magnesium chloride, and zinc chloride. In some embodiments, a chloride salt does not include sodium chloride.

In some embodiments, an ionic compound is potassium chloride. In accordance with various embodiments, potassium chloride may be either agricultural or pharmaceutical grade.

In some embodiments, an aqueous solution used for treating a fish as described herein comprises seawater. In some embodiments, seawater is obtained from ocean. Seawater is composed mostly of six constituents: chloride (Cl⁻), sodium (Na⁺), magnesium (Mg²⁺), sulfate (SO₄²⁻), calcium (Ca²⁺), and potassium (K⁺) (seawater has an average salinity of about 3.5% (35 g/L), which means that every kilogram of seawater has approximately 35 grams of dissolved salts. At this salinity the composition is as follows: (mol/kg) Cl⁻0.546, Na⁺0.469, Mg²⁺ 0.0528, SO₄²⁻ 0.0282, Ca²⁺ 0.0103, K⁺ 0.0102). In some embodiments, seawater used in an aqueous solution is made by dissolving the corresponding salts in freshwater in amounts to provide approximately the same salinity and the same concentration for each of the six ions described above.

In some embodiments, an aqueous solution comprises freshwater. In some embodiments, freshwater is water taken from any natural, non-seawater, source such as lakes, rivers, ponds, and streams.

In some embodiments, an aqueous solution used for removing parasites as described herein comprises an ionic compound at a specific concentration, or range thereof, which causes parasites to detach from an infected fish, without having noticeably negative effects on the fish. In some embodiments, an aqueous solution comprises an ionic compound at a concentration of about 0.2% wt, about 0.15% wt, about 0.1% wt, about 0.08% wt, about 0.06% wt about 0.05% wt, or about 0.02% wt. In some embodiments, an ionic compound is present in an aqueous solution at a concentration of about 100 ppt (parts per thousand) or less. In some embodiments, an ionic compound is present in the aqueous solution at a concentration of about 50 ppt (parts per thousand) or less. In some embodiments, an ionic compound is present in an aqueous solution at a concentration of about 25 ppt or less. In some embodiments, an ionic compound is present in an aqueous solution at a concentration of about 10 ppt or less. In some embodiments, an ionic compound is present in the aqueous solution at a concentration of about 5 ppt or less. In some embodiments, an ionic compound is present in the aqueous solution at a concentration of about 1 ppt or less. In some embodiments, an ionic compound is present in the aqueous solution at a concentration of about 4 ppt, about 3 ppt, about 2 ppt, or about 1 ppt.

In some embodiments, it may be advantageous to include at least one additional mechanism for facilitating removal of a parasite from a fish. Accordingly, in some embodiments, provided methods further include a step of agitating a surface of the fish. In some embodiments, the step of agitating occurs prior to the step of treating the fish with the aqueous solution. In some embodiments, the step of agitating occurs substantially concurrently with the step of treating the fish with the aqueous solution. In some embodiments, the step of agitating occurs after the step of treating the fish with the aqueous solution.

In some embodiments, the agitation of the fish occurs via mechanical means. In some embodiments, the mechanical means are or comprise scrubbing, shaking, and/or rolling. By way of specific example, a step of mechanical treatment as described herein can be brushing the exterior of an infected fish, or rubbing the exterior of the fish.

In some embodiments, the agitation of the fish occurs via a stream of a liquid (e.g., one or more sprays of liquid). In some embodiments, the liquid is seawater, fresh water, the aqueous solution as used in the treatment step, or a mixture thereof. In some embodiments, the agitation occurs via a stream of a gas(e.g., one or more streams of gas). In some embodiments, the gas is air.

In some embodiments, methods described herein include a step of measuring the parasite concentration in a controlled environment into which the fish is placed. One skilled in the art would appreciate that treatment by an aqueous solution as described herein can be considered complete if the benefit of further exposure in the aqueous solution does not balance the potential harm to the fish and/or cost in continuing the step. For example, the treatment can end when further treatment with an aqueous solution as described herein would unlikely remove a significant number of parasites. Accordingly, in some embodiments, a method as described herein comprises monitoring (i.e., measuring at two or more time points) parasite concentration in the controlled environment (e.g., aqueous solution) where the fish is placed in. In some embodiments, a step of treating an infected fish with an aqueous solution comprises placing the fish in a controlled environment for a period of time until the increase of parasite concentration in the aqueous solution slows down or substantially stops.

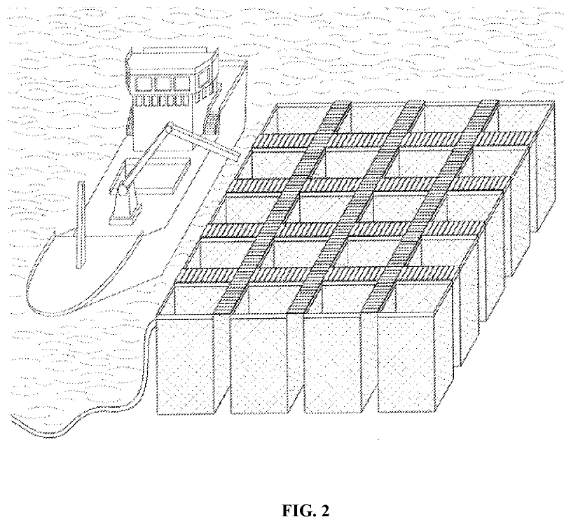

In some embodiments, a method as described herein comprises placing an infected fish in tanks of well boats or fish baths. As shown in FIG. 2, a well boat application is comprised of a boat with a water tight hold or tank on board, where the fish are pumped from a sea cage into the water filled hold or tank. Once the fish are transported into the well, they are treated with the solution for a specified amount of time and returned to the sea cage. A bath application typically involves enclosing an open water cage with a tarpaulin, administering a treatment solution, and allowing the solution to work in an enclosed area for a set amount of time. Once the treatment time has expired the tarp is removed.

In some embodiments, a method as described herein comprises: removing a population of infected fish from a fish farm; placing them in a container containing an aqueous solution as described herein for an appropriate period of time; removing the fish from the container; and returning the fish to the farm.

In some embodiments, a method as described herein comprises: placing a population of infected fish in a tarpaulin in an open water cage; adding an aqueous solution as describe herein to treat the fish for a period as described herein; removing the tarpaulin to let the aqueous solution to mix with the surrounding seawater.

KCl was observed to be more effective on sea lice when used in freshwater than in seawater. Without wishing to be held to a particular theory, a reason for this may be related to the low availability of ions in freshwater for louse osmoregulation. In freshwater, sodium and potassium ions are extremely limited (at least 1,000 times lower than seawater). To compensate in freshwater, sea lice obtain most of their ions from their host (Brandal et al 1976) however the uptake mechanisms for taking up ions in the environment remain functional (although they may be reversed depending on the k-aff value of the ion transporter (Twitchen 1990). Accordingly, by adding KCl to the external environment, it is hypothesized that K ions are rapidly taken up by the louse increasing haemolymph K⁺ levels and raising the haemolymph KCl levels. Given that sodium ions will be diffusing out of the louse during its time in freshwater the ability to compensate for the increase in haemolymph K⁺ ions by increasing sodium is lost hence increasing the efficacy of the treatment. Interestingly this may open up another application for KCl as a treatment for the freshwater parasitic Branchiura parasites Argulus Spp.

Thus, by way of specific example mechanism, aspects of the present invention may function as follows:

Marine arthropods use potassium (K⁺) ions to transmit nervous impulses, mainly as potassium is limiting in seawater, while sodium is not. To achieve this, the axons of sea lice are believed to transport potassium ions into the cell while transporting chloride ions out of the cell maintaining a high potassium/chloride (Cl⁻) ion gradient across the cell membrane.

During depolarization, as the nerve impulse is transmitted, the potassium flows out of the cell very rapidly and chloride ions flow into the cell and hence the cell potential reverses and the impulse is transmitted down the axon to the synapse where it is transmitted by means of the acetylcholine/cholinesterase system to the next nerve fiber or the efferent structure. The ion channels then pump K⁺ back into the cell and Cl⁻ out of the cell allowing the polarity to return to normal.

When lice are treated with KCL, the relative levels of K⁺ ions in the haemolymoh increase (sea lice are osmoconformers and their tissue fluid closely reflects the ion composition in seawater). Having an excess of K⁺ and Cl⁻ in the haemolymph prevents the depolarization of the nerve fiber as K⁺ levels outside the cell always exceed the intracellular concentration and the ion gradient is reversed so K⁺ flow into the cell under all conditions. As the nerve fiber cannot depolarize then nervous transmission is prevented and the animal experiences a flaccid paralysis as muscle fibers are not stimulated to contact.

### Combination Treatment

A method as described herein can be used in conjunction with other methods for removing parasites attached to an infected fish.

In some embodiments, a method as used herein is used in conjunction with an “in-feed” treatment method. In some embodiments, an “in-feed” treatment method is comprised of adding a composition, which is toxic to the sea lice while being harmless to fish, to feed which is then fed to fish.

In some embodiments, a method as used herein is used in conjunction with an “injection” method. In some embodiments, an “injection” method comprises administering in fish a composition, which is toxic to the sea lice while being harmless to fish, by injection.

In various embodiments, provided methods and compositions provide significant advantages over previously known technology for controlling and/or reducing parasite populations in fish populations. For example, previously known technologies include, but are not limited to: use of certain pesticides (e.g., slice pesticide a.k.a. emamectin benzoate), use of hydrogen peroxide, and use of hot water treatments. With regard to use of pesticides, such as emamectin benzoate, there is concern over several aspects, including potential health impacts on consumers (emamectin benzoate can remain in the fish for more than two months), environmental impacts, and the potential for parasites developing resistance. With regard to the use of hydrogen peroxide, there have been significant issues with toxicity, negative environmental impact and complications of subsequent processing steps. With regard to hot water treatments, these are known to significantly increase the cost of farm raising fish as well as exhibiting the potential for negatively impacting the health of the fish. In contrast to all of these treatments, in some embodiments, provided methods and compositions allow for a safe, inexpensive, and easy to manage treatment of aquatic parasites without a significant potential for negative health of environmental impact.

### EXAMPLES

### Example 1

A 0.48% (4.8 g/L-4.8 ppt) pharmaceutical grade KCl compound was tested on 30 Atlantic salmon that were infected with sea lice. The fish and the compound were added to a tank filled with seawater. After 30 minutes 15 fish were removed and placed in a recovery bath of seawater, and at 60 minutes the remaining 15 fish were removed. There were no noticeable adverse effects on the salmon, but there was noticeable sea lice detachment.

### Example 2

A 0.43% % (4.3 g/L-4.3 ppt) agricultural grade KCl compound was tested on Atlantic salmon that were infected with sea lice. After 10 minutes the fish began showing agitation that increased until the 30 minute mark. Fish began losing equilibrium and began to roll over and stop respiring by 20 minutes. The agitation and thrashing of the dying fish detached more lice.

### Example 3

A water holding tank was filled with fresh well water at 10° C.+−2° C. A 0.5% % (5.0 g/L-5.0 ppt) agricultural grade KCl solution was added to the water. A number of farm-raised Atlantic salmon that were infected with sea lice were added to the tank. There was marked sea lice removal. However, within 10 minutes the fish began demonstrating extreme agitation and began to lose equilibrium and their respiration stopped.

### Example 4

A water holding tank was filled with fresh well water at 10° C.+−2° C. A 0.2% % (2.0 g/L-2.0 ppt) agricultural grade KCl solution was added to the water. A number of farm-raised Atlantic salmon that were infected with sea lice were added to the tank. There was marked sea lice detachment after 30 minutes. After 60 minutes, no agitation or adverse effect on the fish was observed.

### Example 5

A pilot laboratory trial was run using various forms of mechanical devices for removal of sea lice from Atlantic salmon (ATS). The effectiveness of the mechanical devices at removing/detaching lice was measured with and without KCl pretreatment, and the results compared.

Methods

The experiment consisted of testing 3 mechanical devices on lice infested Atlantic salmon (ATS) in seawater and then on lice infested Atlantic salmon that had been pre-treated in seawater with KCL at 4 part per thousand (PPT) or 2 PPT for 5 or 15 minutes. The mechanical treatment referred to as Treatment A in Table 1 was a water spray apparatus including multiple jets in a roughly circular arrangement, and treatments B and C in Table 1 were alternative mechanical treatments. Each treatment group was run separately with two fish were staggered by 15 minutes to facilitate counting lice, except for Treatment C as both fish were placed together. The treatment groups were as follows:
1) Treatment A with seawater (2 fish)
2) Treatment A with sea water plus KCL 4 PPT, 15 minutes (2 fish)
3) Treatment B with seawater (2 fish)
4) Treatment B with sea water plus KCL 4 PPT, 15 minutes (2 fish)
5) Treatment A with seawater plus 2PPT, 15 minutes (1 fish)
6) Treatment A with sea water plus KCL 4 PPT, 5 minutes (1 fish)
7) Treatment C with seawater (2 fish)
8) Treatment C sea water plus KCL 2 PPT (2 fish)

Each treatment group was performed essentially as follows:

Pre-Treatment before Treatment A, B, or C
1) Take 1 fish and transfer it to Tank 1 (70 liter tank);
2) Take 1 fish and transfer it to Tank 2, staggered (70 liter tank);
3) Both tanks will be filled with seawater for Round 1 of a treatment group and seawater and KC1 4PPT X for Round 2 for a treatment;
4) Fish swim around freely in treatment Tank 1 for 30 minutes;
5) Fish swim around freely in treatment Tank 2 for 30 minutes;

Estimated Time: 30 minutes +15 minute stagger =45 minutes.

Evaluation of Pretreatment:
1) Remove Fish from Tank 1;
2) Place 1 Fish in Sedation Tank;
3) Count detached lice in Tank 1;
4) Remove 1 Fish from Sedation Tank;
5) Count numbers of Lice on each fish (making sure to differentiate between the two tanks);
6) Repeat for staggered Tank 2.

Perform Mechanical Treatment:
1) Pass fish through mechanical treatments (A, B or C)
2) Count lice on fish

Results

As shown by Table 1, KCl as a pretreatment at 4ppt and 2ppt is effective in removing/detaching lice on its own, and KCl also increases the effectiveness of mechanical treatments A and B at removing/detaching lice. KCl at 4ppt is also more effective that KCl at 2ppt. KCl at 4ppt is more effective at 15 minutes than KCl at 5 minutes. With Treatment C, the number of lice was not counted prior to the treatment.

The results described herein support the use of certain ionic compounds (e.g. KCl), when administered at particular levels or in particular ranges, as potent and safe anti-parasite compounds and as a powerful additives to the fish farming process. In addition, these results support the use of provided ionic compounds as additive treatments for use in conjunction with various means of agitating farm fish.

TABLE 1

| | Fish | Fish | Fish | Fish | Fish | Fish | Fish |
|---|---|---|---|---|---|---|---|
| | #1 | #2 | #3 | #4 | #5 | #6 | #7 |
| Mechanical Treatment | A | A | A | A | B | B | B |
| Seawater | Seawater | Seawater | KCl | KCl | Seawater | Seawater | KCl |
| KCl PPT | NA | NA | 4 | 4 | NA | NA | 4 |
| Treatment time (min) | 15 | 15 | 15 | 15 | 15 | 15 | 15 |
| Sealice counts | | | | | | | |
| Detached in pre-treatment tank | 2 | 5 | 80 | 99 | 12 | 14 | 79 |
| On fish - start of mechanical | 51 | 77 | 28 | 17 | 66 | 82 | 17 |
| treatment | | | | | | | |
| On fish - finish | 13 | 27 | 9 | 1 | 32 | 67 | 8 |
| Total | 53 | 82 | 108 | 116 | 78 | 96 | 96 |
| # removed - mechanical | 38 | 50 | 19 | 16 | 34 | 15 | 9 |
| treatment only | | | | | | | |
| # removed - mechanical + KCl | 40 | 55 | 99 | 115 | 46 | 29 | 88 |
| % removed - mechanical | 74.5% | 64.9% | 67.9% | 94.1% | 51.5% | 18.3% | 52.9% |
| treatment only | | | | | | | |
| % removed - mechanical + KCl | 75.5% | 67.1% | 91.7% | 99.1% | 59.0% | 30.2% | 91.7% |

| | Fish | Fish | Fish | Fish | Fish | Fish | Fish |
|---|---|---|---|---|---|---|---|
| | #8 | #9 | #10 | #11 | #12 | #13 | #14 |
| Mechanical Treatment | B | A | A | C | C | C | C |
| Seawater | KCl | KCl | KCl | Seawater | Seawater | KCl | KCl |
| KCl PPT | 4 | 2 | 4 | NA | NA | 2 | 2 |
| Treatment time (min) | 15 | 15 | 5 | 5 | 5 | 5 | 5 |
| Sealice counts | | | | | | | |
| Detached in pre-treatment tank | 74 | 51 | 52 | N/A | N/A | N/A | N/A |
| On fish - start of mechanical | 17 | 64 | 93 | | | | |
| treatment | | | | | | | |
| On fish - finish | 6 | 25 | 28 | 30 | 14 | 35 | 31 |
| Total | 91 | 115 | 145 | | | | |
| # removed - mechanical | 11 | 39 | 65 | | | | |
| treatment only | | | | | | | |
| # removed - mechanical + KCl | 85 | 90 | 117 | | | | |
| % removed - mechanical | 64.7% | 60.9% | 69.9% | | | | |
| treatment only | | | | | | | |
| % removed - mechanical + KCl | 93.4% | 78.3% | 80.7% | | | | |

1. A method for facilitating removal a parasite attached to a fish comprising
treating the fish with an aqueous solution comprising an ionic compound, wherein the treating does not significantly harm or kill the fish.

2. The method of claim 1, wherein the parasite is selected from the group consisting of: a parasitic crustacean (e.g., a sea louse), a parasitic aquatic arthropod, a Platyhelminthes, and a leech.

3. The method of any one of the preceding claims, wherein the fish is a salmon, carp, tilapia, catfish, halibut, cod, swordfish, haddock, bass, grouper, trout, and/or tuna.

4. The method of any one of the preceding claims, wherein the step of treatment with an aqueous solution comprises placing the fish in the aqueous solution for about 60 minutes or less.

5. The method of any one of the preceding claims, wherein the step of treatment with an aqueous solution comprises placing the fish in the aqueous solution for about 45 minutes or less.

6. The method of any one of the preceding claims, wherein the step of treatment with an aqueous solution comprises placing the fish in the aqueous solution for about 30 minutes or less.

7. The method of any one of the preceding claims, wherein the step of treatment with an aqueous solution comprises placing the fish in the aqueous solution for about 20 minutes or less.

8. The method of any one of the preceding claims, wherein the step of treatment with an aqueous solution comprises placing the fish in the aqueous solution for about 15 minutes or less.

9. The method of any one of the preceding claims, wherein the step of treatment with an aqueous solution is placing the fish in the aqueous solution for about 5 minutes.

10. The method of any one of the preceding claims, wherein the ionic compound is a potassium salt.

11. The method of any one of the preceding claims, wherein the ionic compound is a chloride salt.

12. The method of any one of the preceding claims, wherein the ionic compound is potassium chloride.

13. The method of any one of the preceding claims, wherein the aqueous solution comprises two or more ionic compounds.

14. The method of any one of the preceding claims, wherein the aqueous solution comprises seawater.

15. The method of any one of the preceding claims, wherein the aqueous solution comprises freshwater.

16. The method of any one of the preceding claims, wherein the ionic compound is present in the aqueous solution at a concentration of about 100 ppt or less.

17. The method of any one of the preceding claims, wherein the ionic compound is present in the aqueous solution at a concentration of about 50 ppt or less.

18. The method of any one of the preceding claims, wherein the ionic compound is present in the aqueous solution at a concentration of about 25 ppt or less.

19. The method of any one of the preceding claims, wherein the ionic compound is present in the aqueous solution at a concentration of about 10 ppt or less.

20. The method of any one of the preceding claims, wherein the ionic compound is present in the aqueous solution at a concentration of about 5 ppt or less.

21. The method of any one of the preceding claims, wherein the ionic compound is present in the aqueous solution at a concentration of about 4 ppt.

22. The method of any one of the preceding claims, further comprising a step of agitating a surface of the fish.

23. The method of claim 22, wherein the step of agitating occurs prior to the step of treating the fish with the aqueous solution.

24. The method of claim 22, wherein the step of agitating occurs substantially concurrently with the step of treating the fish with the aqueous solution.

25. The method of claim 22, wherein the step of agitating occurs after the step of treating the fish with the aqueous solution.

26. The method of any one of claims 22-25, wherein the agitation of the fish occurs via mechanical means.

27. The method of claim 26, wherein the mechanical means are or comprise scrubbing, shaking, and/or rolling.

28. The method of any one of claims 22-25, wherein the agitation of the fish occurs via at least one stream of a liquid.

29. The method of claim 28, wherein the liquid is seawater, fresh water, the aqueous solution as used in the treatment step, or a mixture thereof.

30. The method of claim 28 or 29, wherein the agitation occurs via at least one stream of a gas.

31. The method of claim 30, wherein the gas is air.

32. The method of any one of the preceding claims, further comprising
placing the fish in a controlled environment, and
measuring the parasite concentration in the controlled environment both before treatment with the ionic compound and after treatment with the ionic compound.

33. A method for facilitating removal of a parasite attached to a fish comprising treating the fish with an aqueous solution comprising an ionic compound at a concentration of between about 1-5 ppt.

34. The method of claim 33, wherein the ionic compound is or comprises potassium chloride.

35. The method of claim 33 or 34, further comprising a step of agitating a surface of the fish.

36. The method of any one of claims 32-35, wherein the parasite is a sea louse.
